(19) United States
(12) Patent Application Publication
(10) Pub. No.: US 2026/0248969 A1
(43) Pub. Date: Aug. 27, 2026

(54) PEPTIDE MULTIMER PRODUCTS AND METHODS

(71) Applicant: REGENTS OF THE UNIVERSITY OF MICHIGAN, Ann Arbor, MI (US)

(72) Inventors: Thomas D. Wang, Ann Arbor, MI (US); Shuo Feng, Ann Arbor, MI (US)

(21) Appl. No.: 19/162,257

(22) PCT Filed: Mar. 13, 2024
(86) PCT No.: PCT/US24/19730

§ 371 Date: Sep. 4, 2025

Related U.S. Application Data
(60) Provisional application No. 63/490,175, filed on Mar. 14, 2023.

(51) Int. Cl. A61K 49/00; A61K 31/196; A61K 31/282; A61K 31/337; A61K 31/44; A61K 31/4745; A61K 31/7068; A61K 33/243; A61K 47/64; A61K 47/69; A61K 49/14; C07K 7/06; C07K 7/08; C07K 19/00

(52) U.S. Cl. CPC A61K 49/0056; A61K 31/196; A61K 31/282; A61K 31/337; A61K 31/44; A61K 31/4745; A61K 31/7068; A61K 33/243; A61K 47/641; A61K 47/6907; A61K 47/6929; A61K 49/0032; A61K 49/14; C07K 7/06; C07K 7/08; C07K 19/00

(57) ABSTRACT

The disclosure relates to multimers of Glypican-3 (GPC3)-, CD44-, and Epithelial cell adhesion molecule (EpCAM)-specific peptides and the use thereof to detect and treat epithelial cell-derived cancers such as hepatocellular carcinoma (HCC), intrahepatic cholangiocarcinoma (ICC), breast cancer, colon cancer, gastric cancer, ovarian cancer, cervical cancer, and basal cell carcinoma of the skin. The disclosure also relates to methods to monitor the therapeutic response of treated patients using the peptide multimers.

### CROSS-REFERENCE TO RELATED APPLICATIONS

This application claims priority to Provisional Application No. 63/490,175, filed Mar. 14, 2023, which is incorporated herein by reference in its entirety.

### GOVERNMENT SUPPORT

This invention was made with government support under CA230669 awarded by the National Institutes of Health. The government has certain rights in the invention.

### INCORPORATION BY REFERENCE OF THE SEQUENCE LISTING

This application contains, as a separate part of disclosure, a Sequence Listing in computer-readable form (Filename: 58753_SeqListing.XML; 12,136 bytes; Created: Mar. 8, 2023) which is incorporated by reference herein in its entirety.

### FIELD

The disclosure relates to multimers of Glypican-3 (GPC3)-, CD44-, and Epithelial cell adhesion molecule (EpCAM)-specific peptides and the use thereof to detect and treat epithelial cell-derived cancers such as hepatocellular carcinoma (HCC), intrahepatic cholangiocarcinoma (ICC), breast cancer, colon cancer, gastric cancer, ovarian cancer, cervical cancer, and basal cell carcinoma of the skin. The disclosure also relates to methods to monitor the therapeutic response of treated patients using the peptide multimers.

### BACKGROUND

Hepatocellular carcinoma (HCC) accounts for over 840,000 deaths globally and is emerging rapidly as a major contributor to the worldwide healthcare burden. Because few patients are diagnosed early, 5-year survival is <7%, and the median survival length is <1 year [Asrani et al., Burden of liver diseases in the world, 70(1) (2019) 151-171]. In the U.S., the incidence of HCC is rising steadily, and is currently growing faster than any other cancer [Ozakyol, Global Epidemiology of Hepatocellular Carcinoma (HCC Epidemiology). J Gastrointest Cancer 2017; 48:238-2407]. Conventional methods for liver imaging excel at providing anatomical features of masses. Ultrasound is recommended for patients with cirrhosis, but cannot distinguish between malignant and benign lesions. Contrast-enhanced CT and MRI detect HCC based on increased vascularity, but cannot clarify pathology for liver nodules <1-2 cm. Malignant hepatocytes uniquely overexpress targets that can be developed for improved HCC diagnosis and therapy. Thus, early detection of HCC remains a major healthcare challenge globally, and novel diagnostic options are urgently needed.

Tumor heterogeneity poses a substantial challenge for the development of new methods to effectively detect HCC at an early stage. While some progress has been made with serological markers, there have been few advances made with HCC tissue targets, which are critically needed to improve diagnostic and therapeutic strategies. AFP (α-fetoprotein) is used as a serological marker, but has poor tissue sensitivity (~25%).

GPC3 is contemplated herein to be a promising cell surface target for early-stage HCC. The biochemical structure of GPC3 consists of a 70 kD core protein with 2 heparan sulfate side chains, and is anchored via glycosylphosphatidylinositol (GPI) to the cell membrane. GPC3 stimulates the canonical Wnt signaling pathway to promote tumor growth, differentiation, and migration. Increased gene transcription of GPC3 has been found in early stage HCC (<2 cm). On immunohistochemistry, high GPC3 expression was found in small HCC tumors, and increased GPC3 immunostaining was observed in cirrhotic macronodules with malignant potential. GPC3 is markedly overexpressed in HCC, relatively absent in either normal liver or cirrhosis, and has expression levels that reflect tumor stage. Furthermore, mutations in GPC3 and the knockdown of function have been shown to inhibit HCC growth.

CD44 (cluster of differentiation 44) is also contemplated herein to be a tissue biomarker for HCC. The standard isoform is denoted as CD44s, which regulates TGF-β signaling, and spliced variants are denoted as CD44v. Positive staining for CD44 was found on the hepatocyte membrane in up to 84% of early-stage HCC specimens using immunohistochemistry. A smaller percentage was positively identified for CD44 variants.

CD44 is a transmembrane glycoprotein that binds to hyaluronic acid, a component of the extra-cellular matrix and a mediator of fibrogenesis that leads to cirrhosis. CD44 serves as an adhesion molecule, enables communication by cell-cell signal transduction, and regulates a number of biological processes within liver cells. CD44 has various functions in cell division, migration, adhesion, and signaling, and enables cells to interact either through the intracellular matrix or through cellular junctions.

EpCAM (epithelial cell adhesion molecule) is another tissue biomarker contemplated herein for HCC. Up to 77% of HCC specimens stained positive for EpCAM on the hepatocyte surface using immunohistochemistry, and dominant expression appeared in small nodular HCC tumors. EpCAM is a type I transmembrane glycoprotein that functions as an epithelial-specific intercellular cell-adhesion molecule. EpCAM is a direct transcriptional target gene for Wnt-β-catenin signaling in HCC cells, and is considered a biomarker for human epithelial tissues and malignant epithelial tumors. EpCAM functions in cell-cell adhesion, and stimulates cell migration, metastasis, proliferation, and differentiation. EpCAM positive HCC cells possess cancer stem cell traits, including the capacity for self-renewal, differentiation, tumorigenesis, and chemotherapy resistance. Also, EpCAM is contemplated herein to be a promising biomarker for recurrence of HCC.

Image-guided surgery is gaining in popularity with hepatobiliary surgeons. Standard laparoscopes are being adapted to collect NIR fluorescence images for use as an adjunct to conventional white light images. These methodologies enhance image contrast to better locate tumors, identify margins, and detect metastatic lymph nodes. Surgeons currently rely on visual appearance, finger palpation, and intraoperative ultrasound to discriminate between tumor and non-tumor. These techniques are subjective, non-specific for cancer, and prone to inadequate resections and positive margins. By comparison, conventional imaging modalities, including CT, MRI, and PET, are difficult to implement for intra-operative navigation, and intraoperative ultrasound is highly operator dependent. Frozen sections for pathological evaluation obtained intraoperatively from tumor margins is time consuming and not effective for larger lesions.

There remains a need in the art for products and methods for detecting and treating HCC and other epithelial cell-derived cancers, as well as monitoring treatment of patients.

### SUMMARY

The disclosure provides multimers of peptides specific for GPC3, CD44 and EpCAM (herein peptide multimers) and methods to detect and treat epithelial cell-derived cancers including, but not limited to, HCC, ICC, breast cancer, colon cancer, gastric cancer, ovarian cancer, cervical cancer, and basal cell carcinoma of the skin. Moreover, the present disclosure contemplates an imaging methodology utilizing the peptide multimers can visualize specific tumor targets with high contrast in real time and substantially improve clinical outcomes for image-guided surgery. The peptide multimers and methods can also be used monitor the therapeutic response of treated patients.

The disclosure thus provides peptide multimers comprising a peptide specific for GPC3, a peptide specific for CD44 and a peptide specific for EpCAM. The peptide multimer can comprise a tri-lysine linker assembling the three peptides in the multimer.

The GPC3-specific peptide can comprise the amino acids ALLANHEELFQT (SEQ ID NO: 1) (referred to herein as ALL*), ALLANHEELF (SEQ ID NO: 2), GLHTSATNLYLH (SEQ ID NO: 3), SGVYKVAYDWQH (SEQ ID NO: 4), or VGVESCASRCNN (SEQ ID NO: 5). The GPC3-specific peptide can consist essentially of the amino acids ALLANHEELFQT (SEQ ID NO: 1), ALLANHEELF (SEQ ID NO: 2), GLHTSATNLYLH (SEQ ID NO: 3), SGVYKVAYDWQH (SEQ ID NO: 4), or VGVESCASRCNN (SEQ ID NO: 5). The GPC3-specific peptide can consist of the amino acids ALLANHEELFQT (SEQ ID NO: 1), ALLANHEELF (SEQ ID NO: 2), GLHTSATNLYLH (SEQ ID NO: 3), SGVYKVAYDWQH (SEQ ID NO: 4), or VGVESCASRCNN (SEQ ID NO: 5). The disclosure also contemplates an analog of any of those peptides that specifically binds to GPC3.

The CD44-specific peptide can comprise the amino acids WKGWSYLWTQQA (SEQ ID NO: 6) (referred to herein as WKG*). The CD44-specific peptide can consist essentially of the amino acids WKGWSYLWTQQA (SEQ ID NO: 6). The CD44-specific peptide can consist of the amino acids WKGWSYLWTQQA (SEQ ID NO: 6). The disclosure also contemplates an analog of that peptide that specifically binds to CD44.

The EpCAM-specific peptide can comprise the amino acids HPDMFTRTHSHN (SEQ ID NO: 7) (referred to herein as HPD*), HGLHSMHNKLQD (SEQ ID NO: 8), GKPAVHYIHLRH (SEQ ID NO: 9), or HPFLHWNYGQRT (SEQ ID NO: 10). The EpCAM-specific peptide can consist essentially of the amino acids HPDMFTRTHSHN (SEQ ID NO: 7), HGLHSMHNKLQD (SEQ ID NO: 8), GKPAVHYIHLRH (SEQ ID NO: 9), or HPFLHWNYGQRT (SEQ ID NO: 10). The EpCAM-specific peptide can consist of the amino acids HPDMFTRTHSHN (SEQ ID NO: 7), HGLHSMHNKLQD (SEQ ID NO: 8), GKPAVHYIHLRH (SEQ ID NO: 9), or HPFLHWNYGQRT (SEQ ID NO: 10). The disclosure also contemplates an analog of any of those peptides that specifically binds to EpCAM.

The disclosure provides peptide multimers herein that comprise a detectable label. The detectable label can be detectable by optical, photoacoustic, ultrasound, positron emission tomography or magnetic resonance imaging. The label detectable by optical imaging can be fluorescein isothiocyanate (FITC), Cy5, Cy5.5, or IRdye800. The label detectable by magnetic resonance imaging can be gadolinium (Gd) or Gd-DOTA. The detectable label can be attached to a peptide of the multimer by a peptide linker. The terminal amino acid of the linker can be lysine. The linker can comprise the sequence GGGSC. The linker can comprise the sequence GGGSK set out in SEQ ID NO: 11.

The disclosure provides compositions comprising an excipient (such as a pharmaceutically acceptable excipient) and a peptide multimer provided herein.

The disclosure provides methods of using a peptide multimer provided herein for detecting (including, for example, visualizing during image-guided surgery) epithelial cell-derived cancer cells such as HCC cells, ICC cells, breast cancer cells, colon cancer cells, gastric cancer cells, ovarian cancer cells, cervical cancer cells, and basal cell carcinoma of the skin cells.

The disclosure provides methods of using a peptide multimer provided herein for treating epithelial cell-derived cancers including, but not limited to, HCC, ICC, breast cancer, colon cancer, gastric cancer, ovarian cancer, cervical cancer, and basal cell carcinoma of the skin.

The disclosure provides methods of using a peptide multimer provided herein for monitoring the status of epithelial cell-derived cancers such as HCC, ICC, breast cancer, colon cancer, gastric cancer, ovarian cancer, cervical cancer, and basal cell carcinoma of the skin in a patient in which the methods comprise the step of administering a peptide multimer provided herein to the patient, visualizing a first amount of cells labeled with the peptide multimer, and comparing the first amount to a previously-visualized second amount of cells labeled with the peptide multimer, wherein a decrease in the first amount cells labeled relative to the previously-visualized second amount of cells labeled is indicative of effective treatment. The methods can further comprise obtaining a biopsy of the cells labeled by the reagent.

The following Drawings and Detailed Description (including the Examples) illustrate various non-limiting aspects of the subject matter contemplated herein.

### BRIEF DESCRIPTION OF THE DRAWINGS

The patent or application file contains at least one drawing executed in color.

Copies of this patent or patent application publication with color drawing(s) will be provided by the Office upon request and payment of the necessary fee.

FIG. 1 shows peptide multimers for in vivo imaging. A) Monomer peptides, WKGWSYLWTQQA (SEQ ID NO: 1) (WKG*), ALLANHEELFQT (SEQ ID NO: 6) (ALL*), and HPDMFTRTHSHN (SEQ ID NO: 7) (HPD*), specific for CD44, GPC3, and EpCAM, respectively, are arranged in a multimer configuration via a tri-lysine linker. Labeling with Gd-DOTA and IRDye800 provides contrast for either MR or NIR fluorescence imaging, respectively. PEG3 extensions separate the monomer ligands to prevent steric hindrance. B) The biochemical structure for the Gd-DOTA-labeled multimer is shown in 3D to display the relative locations and spatial orientations of the unique monomer peptides. C) Cy5.5-labeled multimer is shown along with D) its biochemical structure in 3D.

FIG. 2 shows multimer validation. Co-localization of binding by IRDye800-labeled multimer and AF488-labeled antibodies specific for A) GPC3, B) CD44, and C) EpCAM to PDOs in vitro is shown. A correlation of p=0.74, 0.80, and 0.75, respectively was measured.

FIG. 3 shown validation of HCC target expression. Strong reactivity (arrow) at the cell surface to antibodies specific for A) GPC3, B) CD44, and C) EpCAM, is seen using IHC. D) Positive staining for cytokeratin confirms presence of human tissues. D) Representative histology (H&E) of PDO shows HCC tumor cells

FIG. 4 shows characterization of IRDye800-labeled peptide monomers. A) WKG*-IRDye800 and anti-CD44-AF488 bind intensely to the surface (arrows) of SK-Hep1 human HCC cells. A Pearson's correlation coefficient of r=0.81 was measured from the merged image. No binding was seen with the scrambled peptide WYK*-IRDye800. B) An apparent dissociation constant of k_{d}=43 nM, R²=0.99, was measured for WKG*-IRDye800 binding to SK-Hep1 cells. The equilibrium dissociation constant k_{d}=1/kₐ was calculated by performing using a least square fit of the data to the non-linear equation I=(I₀+Iₘₐₓkₐ[X])/(I₀+kₐ[X]). I₀ and Iₘₐₓ are the initial and maximum fluorescence intensities, corresponding to no peptide and at saturation, respectively, and [X] represents the concentration of the bound peptide. Results are the mean values from 3 independent experiments. C) Serum stability of WKG*-IRDye800 (200 μM in 200 μL of PBS) in mice (n=5) was measured following intravenous injection. Serum was collected at various time points from 0-24 hours post-injection. The fluorescence intensity I(t) was measured and fit to a first-order kinetics model I(t)=Iₒ+A×exp(−Bt), and a serum half-life of T_{1/2}=5.1 hours was calculated.

FIG. 5 shows orthotopic HCC tumors. A) Ultrasound (US) and B) MR images show the presence of a viable human HCC tumor implanted in the liver of a live mouse. C) White light and D) NIR fluorescence images collected laparoscopically in vivo following intravenous injection of the IRDye800-labeled GPC3 peptide confirm the orthotopic location of the tumor.

FIG. 6 shows in vivo imaging of HCC. A) Peak uptake of the Gd-DOTA-labeled multimer (300 mM in 200 mL PBS) by the orthotopic PDX HCC tumor was observed at 0.5 hours post-injection. A T/B ratio of 1.79±0.03 was measured for the multimer versus 0.99±0.04 for free Gd-DOTA (control) from the same tumor in n=3 mice. B) MR image of HCC tumor at 30 min post-injection is shown. C) NIR fluorescence images collected in vivo are shown from orthotopic HCC PDX tumors after systemic administration of the lead EpCAM peptide HPD*-IRDye800 and control PFH*-IRDye800 (150 μM, 200 μL). D) The intensity for HPD*-IRDye800 was significantly greater than that for PFH*-IRDye800, *P<1×10⁻⁴ by paired t-test.

FIG. 7 shows Gd-DOTA-labeled peptide multimer biodistribution. A) T₁-weighted MR images were collected over time from n=5 mice following intravenous injection of the peptide contrast agent (300 mM in 200 mL PBS). The liver (arrow), spleen (chevron), kidneys (ovals), and bladder (circle) are highlighted in representative images collected at 0.5 hours post-injection (peak uptake). B) The MR signal was observed to peak at 0.5 hours post injection with return to baseline by ~4 hours. The MR intensities were normalized by dividing each value by that at time 0 (pre-injection) for the same mouse and tissues. C) Significantly greater signal was measured from kidney and bladder versus either liver or spleen. An ANOVA with terms for 16 means (4 tissues at 4 time points) was fit to log transformed data. *P<0.01 by pairwise comparisons.

FIG. 8 shows co-localization of IRDye800-labeled peptide multimer and antibody binding. A PDX specimen of human HCC tumor was resected following completion of imaging and was stained with the A) IRDye800-labeled multimer. Adjacent sections were stained with B) anti-GPC3-AF532, C) anti-CD44-AF488, and D) anti-EpCAM-AF594. A Pearson's correlation coefficient of p=0.69, 0.65, and 0.70 was found for the multimer with GPC3, CD44, and EpCAM, respectively, on the E) merged image.

FIG. 9 shows validation of target expression in PDX HCC tumors. Resected PDX specimens were stained for expression of GPC3, CD44, and EpCAM, and regions are shown from A) HCC and B) cirrhosis. Strong 3+reactivity (arrow) was seen for HCC. A standard scoring system was used: 0+, no reactivity or membrane staining in <10% of tumor cells; 1+, a faint/barely perceptible membrane staining is detected in >10% of tumor cells. Cells exhibit incomplete membrane staining; 2+, a weak to moderate complete membrane staining is observed in >10% of tumor cells; 3+, a strong complete membrane staining is observed in >10% of tumor cells.

FIG. 10 shows Cy5.5-labeled peptide multimer binding to HCC cells in vitro. A) The Cy5.5-labeled peptide multimer showed strong binding to the cell surface (arrow) of human CCA-156 HCC cells while minimal signal was seen with the peptide monomers ALL*-Cy5.5, WKG*-Cy5.5, and HPD*-Cy5.5, specific for GPC3, CD44, and EpCAM, respectively. B) Quantified fluorescence intensities showed a 5.48, 1.84 and 2.00-fold increase for the multimer versus the GPC3, CD44, and EpCAM peptide monomers, respectively.

FIG. 11 shows immunofluorescence of HCC patient-derived organoids (PDOs). Co-localization was seen for binding by Cy5.5-labeled multimer and AF488-labeled antibodies specific for A) GPC3, B) CD44, and C) EpCAM to patient-derived HCC organoids. A correlation of r=0.74, 0.80, and 0.75, respectively, was measured on the merged images.

FIG. 12 shows immunohistochemistry of HCC PDOs. Strong reactivity (arrow) was seen with antibodies specific for A) GPC3, B) CD44, and C) EpCAM to validate target expression. D) Representative histology (H&E) shows HCC tumor cells. Positive staining with E) anti-Hep-Par1 and F) anti-cytokeratin confirmed human and liver specific tissues, respectively.

FIG. 13 shows in vivo MR imaging using Gd-DOTA-labeled peptide multimer. T₁-weighted MR images are shown for Gd-DOTA-labeled A) peptide multimer, B) GPC3-, C) CD44-, and D) EpCAM-specific peptide monomers, and E) free Gd-DOTA. Images were collected at pre, 0.5, 1, 1.5, 2, and 4 hours post-injection from mice bearing orthotopic HCC PDX tumors ~3 mm in dimension using a concentration of 300 μM in 200 μL PBS. F) Peak uptake occurred at 30 min post-injection, and cleared after ~4 hours. G) The target-to-background (T/B) ratio for the multimer was greater than that for the peptide monomers and free Gd-DOTA (n=5 animals per group).

FIG. 14 shows in vivo binding competition. Blocking was performed to compete for binding by administering unlabeled peptide multimer and peptide monomers ALL*, WKG*, and HPD*, specific for GPC3, CD44, and EpCAM, respectively, prior to the Gd-DOTA-labeled peptide multimer. Concentrations of 1.5 mM in 100 mL of PBS were used. The target-to-background (T/B) ratio from the orthotopic HCC PDX tumors was reduced for each group.

FIG. 15 shows Gd-DOTA-labeled peptide multimer biodistribution. A,B) T₁-weighted MR images were collected to evaluate uptake of the Gd-DOTA labeled peptide multimer by major organs, including kidney, liver, and spleen. MR signal in kidney peaked at 1.5 hour post-injection and returned to baseline by ~4 hours.

FIG. 16 shows Gd-DOTA-labeled peptide multimer stability. Serum stability was measured using analytical HPLC at time points ranging from 0-24 hours. A half-life of T_{1/2}=2.6 hours was measured, R²=0.98.

FIG. 17 shows immunofluorescence of a human HCC PDX tumor specimen stained with Cy5.5-labeled peptide multimer. A human HCC PDX tumor specimen was stained following completion of imaging and with A) Cy5.5-labeled peptide multimer. Adjacent sections were stained with B) anti-GPC3-AF532 antibody, C) anti-CD44-AF488 antibody, and D) anti-EpCAM-AF594 antibody. E) Co-localization of binding is shown on the merged image. A Pearson's correlation coefficient of p=0.69, 0.65, and 0.70 was measured for the multimer and the GPC3, CD44, and EpCAM antibodies, respectively.

FIG. 18 shows animal necropsy. Healthy mice were sacrificed at 48 hours post-injection with Gd-DOTA-labeled peptide multimer (300 μM, 200 μL). No signs of acute toxicity were seen on histology (H&E) of vital organs, including A) brain, B) heart, C) liver, D) spleen, E) lung, F) kidney, G) stomach, and H) intestine.

FIG. 19 shows animal toxicology. Healthy mice were sacrificed at 48 hours post-injection with Gd-DOTA-labeled peptide multimer (300 μM, 200 μL). No signs of acute toxicity were seen.

FIG. 20 shows specific peptide multimer binding to human liver specimens. A) Cy5.5-labeled peptide multimer (red), anti-GPC3-AF532 (yellow), anti-CD44-AF488 (green), and anti-EpCAM-AF594 (purple) were used to stain human liver specimens of A) HCC, B) cirrhosis, C) adenoma, and D) normal arranged in a tissue microarray. Co-localization of peptide multimer and antibody binding is shown on the merged image. A Pearson's correlation coefficient of p=0.76, 0.70, and 0.81, respectively, p=0.69, 0.60, and 0.66, respectively, p=0.82, 0.74, and 0.83, respectively, and p=0.74, 0.69, and 0.83, respectively, was measured for GPC3, CD44, and EpCAM, respectively, from the merged image of HCC, cirrhosis, adenoma, and normal liver, respectively. E) Quantified fluorescence intensities for HCC were greater versus cirrhosis, adenoma, and normal with mean±SD values of 8.4±1.0, 6.4+0.7, 5.8±0.4, and 6.4±0.4, respectively. A total of n=120 specimens were evaluated. E,F) ROC curves show a sensitivity of 87% and 90% with a specificity of 80% and 80% for the multimer to distinguish HCC from cirrhosis and from non-HCC (cirrhosis, adenoma, and normal), respectively, with an AUC=0.89 and 0.90, respectively.

### DETAILED DESCRIPTION

### Peptides and Multimers

### GPC3-Specific Peptides

The disclosure provides GPC3-specific peptides. For example, the GPC3-specific peptide can comprise the amino acids ALLANHEELFQT (SEQ ID NO: 1) (ALL*), ALLANHEELF (SEQ ID NO: 2), GLHTSATNLYLH (SEQ ID NO: 3), SGVYKVAYDWQH (SEQ ID NO: 4), or VGVESCASRCNN (SEQ ID NO: 5). The GPC3-specific peptide can consist essentially of the amino acids ALLANHEELFQT (SEQ ID NO: 1) (ALL*), ALLANHEELF (SEQ ID NO: 2), GLHTSATNLYLH (SEQ ID NO: 3), SGVYKVAYDWQH (SEQ ID NO: 4), or VGVESCASRCNN (SEQ ID NO: 5). The GPC3-specific peptide can consist of the amino acids ALLANHEELFQT (SEQ ID NO: 1) (ALL*), ALLANHEELF (SEQ ID NO: 2), GLHTSATNLYLH (SEQ ID NO: 3), SGVYKVAYDWQH (SEQ ID NO: 4), or VGVESCASRCNN (SEQ ID NO: 5). The disclosure also contemplates an analog of any of those peptides that specifically binds to GPC3. The disclosure also contemplates peptides that compete with peptides provided herein for binding to GPC3.

### CD44-Specific Peptides

The disclosure provides CD44-specific peptides. For example, the CD44-specific peptide can comprise the amino acids WKGWSYLWTQQA (SEQ ID NO: 6) (WKG*). The CD44-specific peptide can consist essentially of the amino acids WKGWSYLWTQQA (SEQ ID NO: 6) (WKG*). The CD44-specific peptide can consist of the amino acids WKGWSYLWTQQA (SEQ ID NO: 6) (WKG*). The disclosure also contemplates an analog of that peptide that specifically binds to CD44. The disclosure also contemplates peptides that compete with the peptide provided herein for binding to CD44.

### EpCAM-Specific Peptides

The disclosure provides EpCAM-specific peptides. For example, the EpCAM-specific peptide can comprise the amino acids HPDMFTRTHSHN (SEQ ID NO: 7) (HPD*), HGLHSMHNKLQD (SEQ ID NO: 8), GKPAVHYIHLRH (SEQ ID NO: 9), or HPFLHWNYGQRT (SEQ ID NO: 10). The EpCAM-specific peptide can consist essentially of the amino acids HPDMFTRTHSHN (SEQ ID NO: 7) (HPD*), HGLHSMHNKLQD (SEQ ID NO: 8), GKPAVHYIHLRH (SEQ ID NO: 9), or HPFLHWNYGQRT (SEQ ID NO: 10). The EpCAM-specific peptide can consist of the amino acids HPDMFTRTHSHN (SEQ ID NO: 7) (HPD*), HGLHSMHNKLQD (SEQ ID NO: 8), GKPAVHYIHLRH (SEQ ID NO: 9), or HPFLHWNYGQRT (SEQ ID NO: 10). The disclosure also contemplates an analog of any of those peptides that specifically binds to EpCAM. The disclosure also contemplates peptides that compete with peptides provided herein for binding to EpCAM.

The phrases “specific for,” “specifically binds to” or “specifically detects” mean that a peptide or peptide multimer binds to and is detected in association with its target on a cell, and the peptide or multimer does not bind to and is not detected in association with another target on the cell at the level of sensitivity at which the method is carried out.

### Peptide Multimers

The “peptide multimers” provided herein comprise three peptides: a GPC3-specific peptide, a CD44-specific peptide and an EpCAM-specific peptide. The GPC3-specific peptide of a multimer can be, for example, a peptide of SEQ ID NOs: 1-5. The CD44-specific peptide of a multimer can be, for example, a peptide of SEQ ID NO: 6. The EpCAM-specific peptide of a multimer can be, for example, a peptide of SEQ ID NO: 7-10.

A peptide multimer can comprise at least one detectable label as a moiety attached to a peptide provided herein. The detectable label can be detected, for example, by optical, ultrasound, PET, SPECT, or magnetic resonance imaging. The label detectable by optical imaging can be, for example, fluorescein isothiocyanate (FITC), Cy5, Cy5.5 or IRdye800 (also known as IR8000W). The label detectable by magnetic resonance imaging can be, for example, gadolinium, Gd-DOTA or an iron oxide nanoparticle. More detectable labels contemplated are set out below.

A detectable label can be attached to a peptide multimer provided herein. The terminal amino acid of the linker can be a lysine such as in the exemplary linker GGGSK (SEQ ID NO: 11).

A peptide multimer can comprise at least one therapeutic moiety attached to the peptide multimer. The therapeutic moiety can be a chemopreventative or chemotherapeutic agent. For example, the chemopreventative agent can be sorafenib. For example, the chemopreventative agent can be celecoxib. As other non-limiting examples, the chemotherapeutic agent can be carboplatin, paclitaxel, cisplatin, 5-fluorouracil (5-FU), oxaliplatin, capecitabine, chloambucil or irinotecan. The therapeutic moiety can be a nanoparticle or micelle encapsulating another therapeutic moiety. For example, sorafenib, celecoxib, carboplatin, paclitaxel, cisplatin, 5-fluorouracil (5-FU), oxaliplatin, capecitabine, chloambucil, or irinotecan can be encapsulated. More therapeutic moieties contemplated are set out below.

A peptide conjugate can comprise at least one detectable label attached to the peptide multimer, and at least one therapeutic moiety attached to the peptide multimer.

### Linkers, Peptides and Peptide Analogs

As used herein, a “linker” is a sequence of amino acids located at the C-terminus of a peptide of the disclosure. The linker sequence can terminate with a lysine residue.

The presence of a linker can result in at least a 1% increase in detectable binding of a peptide multimer provided herein to cells compared to the detectable binding of the peptide multimer in the absence of the linker. The increase in detectable binding can be at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 100-fold or more.

The term “peptide” refers to molecules of 2 to 50 amino acids, molecules of 3 to 20 amino acids, and those of 6 to 15 amino acids. Peptides and linkers contemplated herein can be 5 amino acids in length. A polypeptide or linker can be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length.

Various scaffolds are known in the art upon which three peptides can be presented in multimeric form. Three peptides can be presented in multimer form on a trilysine dendritic wedge. Other scaffolds known in the art include, but are not limited to, other dendrimers and polymeric (e.g., PEG) scaffolds.

It will be understood that peptides and linkers provided herein optionally incorporate modifications known in the art and that the location and number of such modifications are varied to achieve an optimal effect in the peptide and/or linker analog.

A peptide analog having a structure based on one of the peptides disclosed herein (the “parent peptide”) can differ from the parent peptide in one or more respects. Accordingly, as appreciated by one of ordinary skill in the art, the teachings regarding the parent peptides provided herein can also be applicable to the peptide analogs.

A peptide analog can comprise one or more D amino acids to increase the resistance of the peptides to proteases to increase serum stability.

The peptide analog can comprise the structure of a parent peptide, except that the peptide analog comprises one or more non-peptide bonds in place of peptide bond(s). The peptide analog can comprise in place of a peptide bond, an ester bond, an ether bond, a thioether bond, an amide bond, and the like. The peptide analog can be a depsipeptide comprising an ester linkage in place of a peptide bond.

The peptide analog can comprise the structure of a parent peptide described herein, except that the peptide analog comprises one, two, three, four or more amino acid substitutions, e.g., one, two, three, four or more conservative amino acid substitutions.

Conservative amino acid substitutions are known in the art, and include amino acid substitutions in which one amino acid having certain physical and/or chemical properties is exchanged for another amino acid that has the same chemical or physical properties. For instance, the conservative ammo acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, lie, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

The peptide analog can comprise one, two, three, four or more synthetic amino acids, e.g., an amino acid non-native to a mammal. Synthetic amino acids include β-alanine (β-Ala), N-Q-methyl-alanine (Me-Ala), aminobutyric acid (Abu), γ-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ Glu), homoserine (Hse), hydroxyproline (Hyp), isoleucine N-methoxy-N-methyl amide, methyl-isoleucine (Melle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O₂)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-CI)), 4-fluorophenylalanine (Phe(4-F)), 4-nitrophenylalanine (Phe(4-NO₂)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), O-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), O-benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), and alkylated 3-mercaptopropionic acid.

The peptide analog can comprise one, two, three, four or more non-conservative amino acid substitutions and the peptide analog still functions to a similar extent, the same extent, or an improved extent as the parent peptide. The peptide analog can comprise one or more non-conservative amino acid substitutions exhibits about the same or greater binding to HCC cells in comparison to the parent peptide.

The peptide analog can comprise one, two, three, four or more amino acid insertions or deletions, in comparison to the parent peptide described herein. The peptide analog can comprise an insertion of one or more amino acids in comparison to the parent peptide. The peptide analog can comprise a deletion of one or more amino acids in comparison to the parent peptide. The peptide analog can comprise an insertion of one or more amino acids at the N- or C-terminus in comparison to the parent peptide. The peptide analog can comprise a deletion of one or more amino acids at the N- or C-terminus in comparison to the parent peptide.

Peptide analogs provided can exhibit about the same or greater binding to its target as the original peptide.

The peptides and peptide analogs provided herein can be PEGylated or acetylated to improve serum stability.

### Detectable Labels

As used herein, a “detectable label” is any label that can be used to identify the binding of a composition of the disclosure to target cells. Non-limiting examples of detectable labels are fluorophores, chemical or protein tags that enable the visualization of a polypeptide. Visualization in certain aspects is carried out with the naked eye, or a device (for example and without limitation, an endoscope) and can also involve an alternate light or energy source.

Fluorophores, chemical and protein tags that are contemplated for use herein include, but are not limited to, FITC, Cy5, Cy 5.5, Cy 7, Li-Cor, a radiolabel, biotin, luciferase, 1,8-ANS (1-Anilinonaphthalene-8-sulfonic acid), 1-Anilinonaphthalene-8-sulfonic acid (1,8-ANS), 5-(and-6)-Carboxy-2′, 7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody multimer pH 7.2, Alexa Fluor 488 antibody multimer pH 8.0, Alexa Fluor 488 hydrazide-water, Alexa Fluor 532 antibody multimer pH 7.2, Alexa Fluor 555 antibody multimer pH 7.2, Alexa Fluor 568 antibody multimer pH 7.2, Alexa Fluor 610 R-phycoerythrin streptavidin pH 7.2, Alexa Fluor 647 antibody multimer pH 7.2, Alexa Fluor 647 R-phycoerythrin streptavidin pH 7.2, Alexa Fluor 660 antibody multimer pH 7.2, Alexa Fluor 680 antibody multimer pH 7.2, Alexa Fluor 700 antibody multimer pH 7.2, Allophycocyanin pH 7.5, AMCA multimer, Amino Coumarin, APC (allophycocyanin),Atto 647, BCECF pH 5.5, BCECF pH 9.0, BFP (Blue Fluorescent Protein), Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody multimer pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, C5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, Dil, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody multimer pH 8.0, Erythrosin-5-isothiocyanate pH 9.0, eYFP (Enhanced Yellow Fluorescent Protein), FDA, FITC antibody multimer pH 8.0, FlAsH, Fluo-3, Fluo-3 Ca2+, Fluo-4, Fluor-Ruby, Fluorescein, Fluorescein 0.1 M NaOH, Fluorescein antibody multimer pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Ca, Fura-2 Ca2+, Fura-2, Fura-2, GFP (S65T), HcRed, Indo-1 Ca2+, Indo-1, Ca free, Indo-1, Ca saturated, IDRdye800 (IR800CW), JC-1, JC-1 pH 8.2, Lissamine rhodamine, Lucifer Yellow, CH, Magnesium Green, Magnesium Green Mg2+, Magnesium Orange, Marina Blue, mBanana, mCherry, mHoneydew, mOrange, mPlum, mRFP, mStrawberry, mTangerine, NBD-X, NBD-X, MeOH, NeuroTrace 500/525, green fluorescent Nissl stain-RNA, Nile Blue, Nile Red, Nile Red-lipid, Nissl, Oregon Green 488, Oregon Green 488 antibody multimer pH 8.0, Oregon Green 514, Oregon Green 514 antibody multimer pH 8.0, Pacific Blue, Pacific Blue antibody multimer pH 8.0, Phycoerythrin, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody multimer pH 8.0, Rhodamine Green pH 7.0, Rhodol Green antibody multimer pH 8.0, Sapphire, SBFI-Na⁺, Sodium Green Na⁺, Sulforhodamine 101, Tetramethylrhodamine antibody multimer pH 8.0, Tetramethylrhodamine dextran pH 7.0, and Texas Red-X antibody multimer pH 7.2.

Non-limiting examples of chemical tags contemplated herein include radiolabels.

For example and without limitation, radiolabels that contemplated in the compositions and methods of the present disclosure include ¹¹C, ¹³N, ¹⁵O, ¹⁸F, ³²P, ⁵²Fe, ⁶²Cu, ⁶⁴Cu, ⁶⁷Cu, ⁶⁷Ga, ⁶⁸Ga, ⁸⁶Y, ⁸⁹Zr, ⁹⁰Y, ⁹⁴mTc, ⁹⁴Tc, ⁹⁵Tc, ⁹⁹mTc, ¹⁰³Pd, ¹⁰⁵Rh, ¹⁰⁹Pd, ¹¹¹Ag, ¹¹¹In, ¹²³I, ¹²⁴I, ¹²⁵I, ¹³¹I, ¹⁴⁰La, ¹⁴⁹Pm, ¹⁵³Sm, ¹⁵⁴⁻¹⁵⁹Gd, ¹⁶⁵Dy, ¹⁶⁶Dy, ¹⁶⁶Ho, ¹⁶⁹Yb, ¹⁷⁵Yb, ¹⁷⁵Lu ¹⁷⁷Lu, ¹⁸⁶Re, ¹⁸⁸Re, ¹⁹²|r ¹⁹⁸Au, ¹⁹⁹Au, and ²¹²Bi.

For magnetic resonance imaging, non-limiting examples of detectable labels contemplated herein are gadolinium (Gd), Gd-DOTA and iron oxide nanoparticles.

For positron emission tomography (PET) tracers including, but not limited to, carbon-11, nitrogen-13, oxygen-15 and fluorine-18 are used.

A worker of ordinary skill in the art will appreciate that there are many such detectable labels that can be used to visualize a target on a cell, in vitro, in vitro or ex vivo.

### Therapeutic Moieties

Therapeutic moieties contemplated herein include, but are not limited to polypeptides (including protein therapeutics) or peptides, small molecules, chemotherapeutic agents, or combinations thereof.

The term “small molecule”, as used herein, refers to a chemical compound, for instance a peptidometic or oligonucleotide that can optionally be derivatized, or any other low molecular weight organic compound, either natural or synthetic.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

The therapeutic moiety can be a protein therapeutic. Protein therapeutics include, without limitation, cellular or circulating proteins as well as fragments and derivatives thereof. Still other therapeutic moieties include polynucleotides, including without limitation, protein coding polynucleotides, polynucleotides encoding regulatory polynucleotides, and/or polynucleotides which are regulatory in themselves. Optionally, the compositions comprise a combination of the compounds described herein.

Protein therapeutics can include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β33, transforming growth factor β5, latent transforming growth factor β1, transforming growth factor β binding protein I, transforming growth factor β binding protein II, transforming growth factor β binding protein Ill, tumor necrosis factor receptor type I, tumor necrosis factor receptor type II, urokinase-type plasminogen activator receptor, vascular endothelial growth factor, and chimeric proteins and biologically or immunologically active fragments thereof.

Therapeutic moieties can also include chemotherapeutic agents. A chemotherapeutic agent contemplated for use in a peptide conjugate provided herein includes, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, capecitabine, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural conjugates including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinium coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; topoisomerase inhibitors such as irinotecan; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Chemotherapeutic agents such as gefitinib, sorafenib and erlotinib are also specifically contemplated.

Therapeutic moieties to be attached to a peptide described herein also include nanoparticles or micelles that, in turn, encapsulate another therapeutic moiety. The nanoparticles can be polymeric nanoparticles such as described in Zhang et al., ACS NANO, 2(8): 1696-1709 (2008) or Zhong et al., Biomacromolecules, 15:1955-1969 (2014). The micelles can be polymeric micelles such as octadecyl lithocholate micelles described in Khondee et al., J. Controlled Release, 199:114-121 (2015) and WO 2017/096076 (published Jun. 8, 2017). The peptide multimers comprising nanoparticles or micelles can encapsulate, for example, sorafenib, celecoxib, carboplatin, paclitaxel, cisplatin, 5-fluorouracil (5-FU), oxaliplatin, capecitabine, chloambucil, or irinotecan.

### Compositions

The disclosure provides a composition comprising at least one peptide multimer provided herein and a pharmaceutically acceptable excipient.

### Methods

The disclosure provides methods for specifically detecting epithelial cell-derived cancers cells such as HCC cells, ICC cells, breast cancer cells, colon cancer cells, gastric cancer cells, ovarian cancer cells, cervical cancer cells, and basal cell carcinoma of the skin cells in a patient comprising the steps of administering a peptide multimer provided herein comprising a detectable label to the patient and detecting binding of the peptide multimer to a target (e.g., a protein such as GPC3, CD44 or EpCAM) on the cells. Such methods can be used, for example, to determine the presence of epithelial cell-derived cancers in a patient. Another example of use of such methods is visualizing epithelial cell-derived cancer cells during image-guided surgery.

Methods provided herein can comprise the acquisition of a tissue sample from a patient. The tissue sample can be a tissue or organ of said patient.

The disclosure provides a method for delivering a therapeutic moiety to a patient comprising the step of administering a peptide multimer provided herein comprising the therapeutic moiety to the patient.

The disclosure provides a method for treating an epithelial cell-derived cancer (such as HCC, ICC, breast cancer, colon cancer, gastric cancer, ovarian cancer, cervical cancer and basal cell carcinoma of the skin) in a patient comprising the step of administering a peptide multimer provided herein comprising a therapeutic moiety to the patient.

The disclosure provides a method of determining the effectiveness of a treatment for epithelial cell-derived cancers (such as ICC, HCC, breast cancer, colon cancer gastric cancer, ovarian cancer, cervical cancer and basal cell carcinoma of the skin) in a patient comprising the step of administering a peptide multimer provided herein comprising a detectable label to the patient, visualizing a first amount of cells labeled with the peptide multimer, and comparing the first amount to a previously-visualized second amount of cells labeled with the peptide multimer, wherein a decrease in the first amount cells labeled relative to the previously-visualized second amount of cells labeled is indicative of effective treatment. A decrease of 5% can be indicative of effective treatment. A decrease of about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more can indicative of effective treatment. The method can further comprise obtaining a biopsy of the cells labeled by the peptide multimer.

Methods provided herein can be used for primary, secondary or recurrent cancers.

Peptide multimers and compositions thereof provided herein can be delivered by any route that effectively reaches target cells (e.g., cancer cells) in a patient including, but not limited to, administration by an intravenous, topical, oral or nasal route.

The disclosure provides a kit for administering a composition provided herein to a patient in need thereof, where the kit comprises a composition provided herein, instructions for use of the composition and a device for administering the composition to the patient.

### Dosages

Dosages of a peptide multimer provided herein are administered as a dose measured in, for example, mg/kg. Contemplated mg/kg doses include, but are not limited to, about 1 mg/kg to about 60 mg/kg. Illustrative specific ranges of doses in mg/kg include about 1 mg/kg to about 20 mg/kg, about 5 mg/kg to about 20 mg/kg, about 10 mg/kg to about 20 mg/kg, about 25 mg/kg to about 50 mg/kg, and about 30 mg/kg to about 60 mg/kg. The precise effective amount for a subject will depend upon the subject's body weight, size, and health; the nature and extent of the condition; and the therapeutic or combination of therapeutics selected for administration. Effective amounts for a given situation can be determined by routine experimentation that is within the skill and judgment of the clinician.

“Effective amount” as used herein refers to an amount of a peptide multimer provided herein sufficient to visualize the identified disease or condition, or to exhibit a detectable therapeutic effect. That is, the effect is detected by, for example, an improvement in clinical condition or reduction in symptoms. The precise effective amount for a subject will depend upon the subject's body weight, size, and health, as well as the nature and extent of the condition and the therapeutic or combination of therapeutics selected for administration. Effective amounts for a given situation can be determined by routine experimentation that is within the skill and judgment of the clinician.

### Formulations

Compositions provided herein comprise pharmaceutically acceptable excipients such as carriers, solvents, stabilizers, adjuvants, diluents, etc., depending upon the particular mode of administration and dosage form. The compositions are generally formulated to achieve a physiologically compatible pH, and range from a pH of about 3 to a pH of about 11, about pH 3 to about pH 7, depending on the formulation and route of administration. The pH can be adjusted to a range from about pH 5.0 to about pH 8. The compositions can comprise a therapeutically effective amount of at least one peptide or peptide multimer as described herein, together with one or more pharmaceutically acceptable excipients. Optionally, the compositions comprise a combination of the compounds described herein, or can include a second active ingredient useful in the treatment or prevention of bacterial growth (for example and without limitation, anti-bacterial or anti-microbial agents), or can include a combination of peptide multimers provided herein.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol) wetting or emulsifying agents, pH buffering substances, and the like.

### Other Terminology and Disclosure

As used herein and in the appended claims, the singular forms “a,” “and,” and “the” include plural referents unless the context clearly dictates otherwise. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure.

All publications mentioned herein are incorporated herein by reference to disclose and describe the methods and/or materials for the purpose for which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This disclosure is intended to provide support for all such combinations.

As used herein, “contemplated,” “may,” “may comprise,” “may be,” “can,” “can comprise” and “can be” all indicate something envisaged by the inventors that is functional and available as part of the subject matter provided.

### EXAMPLES

While the following examples describe specific embodiments, variations and modifications will occur to those skilled in the art. Accordingly, only such limitations as appear in the claims should be placed on the invention.

Monomer peptides herein are arranged in a multimer configuration to produce multivalent ligand-target interactions. Increased sensitivity occurs from simultaneous detection of multiple targets. Greater specificity arises from the multimer binding to a larger combined target epitope. Cancer targets may be detected at lower levels of expression, and at an earlier time point.

Optimized monomer peptides specific for early HCC tissue targets were arranged in a multimer configuration and were labeled with either Gd-DOTA or IRDye800 for MR and NIR fluorescence imaging, respectively. Specific target binding was validated in vitro and in vivo using patient-derived organoids (PDO) and a patient-derived xenograft (PDX) model of HCC, respectively.

### Example 1

### Configuration of Peptide Multimer for In Vivo Imaging

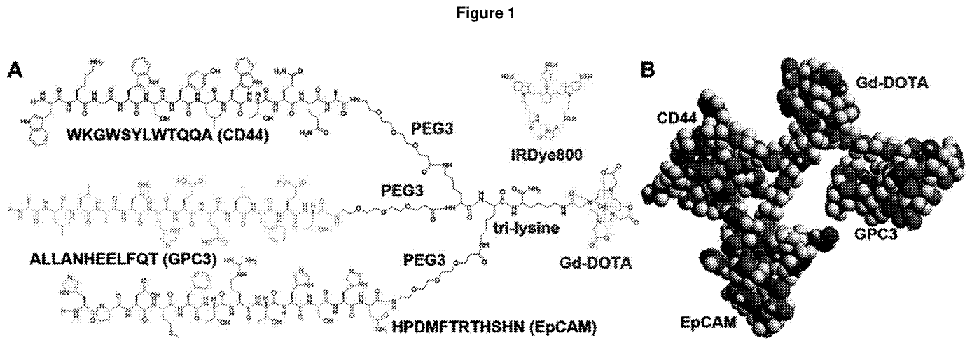

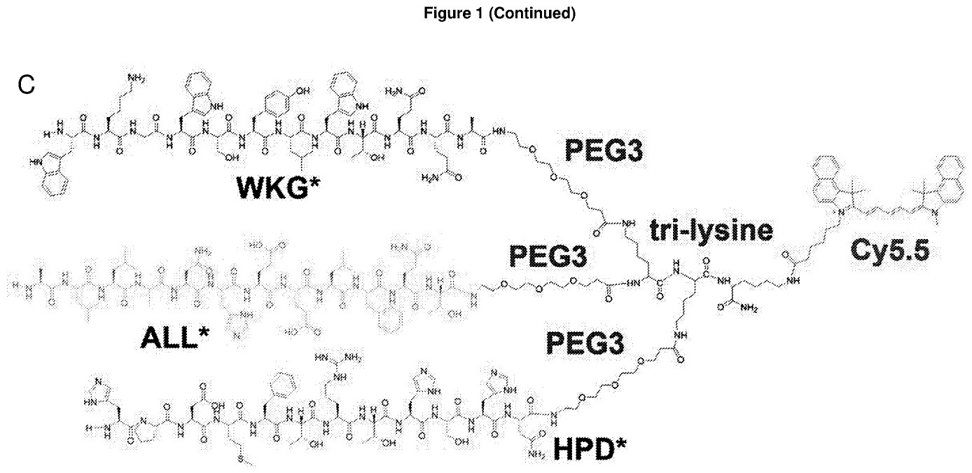

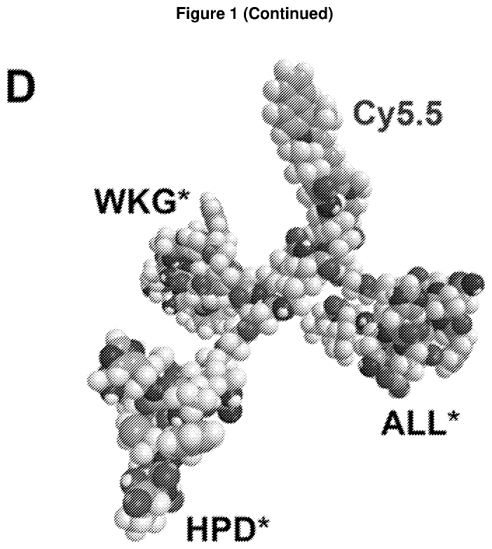

Optimized GPC3-, CD44-, and EpCAM-specific peptide monomers were arranged in a multimer configuration via a tri-lysine linker and PEG3 linkers, and were labeled with Gd-DOTA and IRDye800. See FIG. 1A. A 3D biochemical structure shows the relative spacing and distance between the unique peptides in FIG. 1B. A peptide multimer labeled with Cy5.5 (shown in FIG. 1C,D) was also generated. The IRDye800-labeled multimer was used first to validate specific target binding using NIR fluorescence imaging. The Gd-DOTA-labeled multimer was then used to characterize in vivo tumor uptake using MR imaging.

### Example 2

Validation with Patient-Derived Organoids (PDOs)

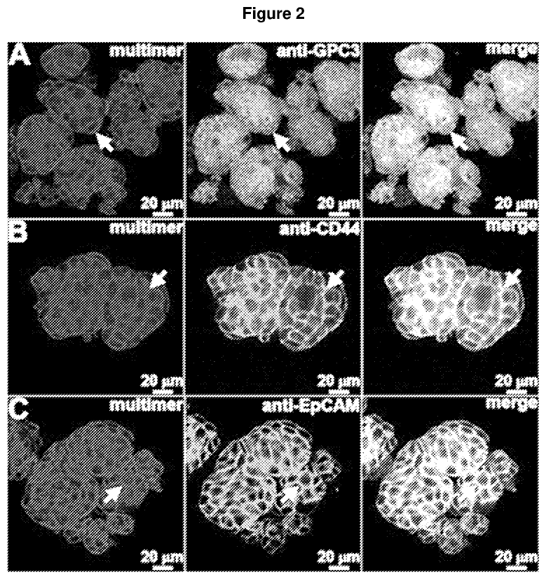

PDOs were established from human HCC specimens. These immortalized human tissues provided clinically relevant target expression levels and genetic heterogeneity seen in the HCC patient population. Short tandem repeat analysis was performed to authenticate human genomic expression and characterize genetic variations. Fluorescence images from a representative HCC organoid are shown in FIG. 2. Images collected using the candidate IRDye800-labeled multimer, AF488-labeled monoclonal antibodies specific for GPC3, CD44, and EpCAM, and merged are shown, FIG. 2A-C. Strong fluorescence intensity can be seen at the cell surface (arrows). Multimer and antibody co-localization was measured using correlation analysis.

### Example 3

Validation of HCC PDO Target Expression with Immunohistochemistry

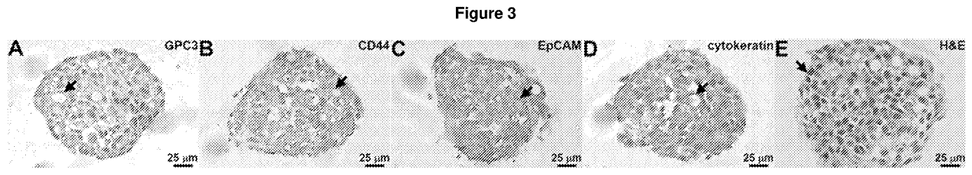

Expression of early-stage HCC tissue targets by each newly established PDO were validated using immunohistochemistry (IHC). Results from a current PDO using monoclonal antibodies specific for GPC3, CD44, and EpCAM are shown in FIG. 3A-C. Strong reactivity can be appreciated at the cell surface (arrows) for each target. A positive cytokeratin stain confirmed the presence of human tissues, FIG. 3D. Representative histology (H&E) shows presence of HCC tumor cells (arrow) in the PDO, FIG. 3E.

### Example 4

Validation of Peptide Monomer with Co-Localization

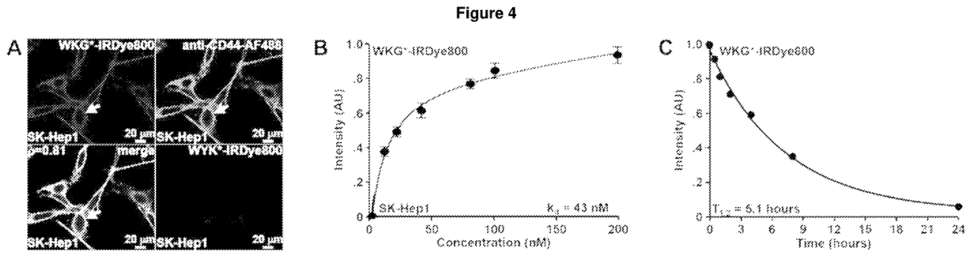

Specific binding of the optimized IRDye800-labeled WKD* monomer peptide was substantiated using a co-localization of binding to known antibodies. Human Hep3B, HepG2, and Sk-Hep1 HCC cells that overexpress early-stage HCC targets were used. A Pearson's correlation coefficient ρ was calculated from fluorescence intensities on merged images. Measurements were made from n=10 cells chosen randomly from 3 slides. Data for CD44 is shown in FIG. 4A, and similar data was obtained for GPC3 and EpCAM. As shown in the figure, binding by WKG*-IRDye800 and anti-CD44-AF488 to the surface (arrows) of Sk-Hep1 cells co-localized with a correlation of p=0.81 measured on the merged image.

### Example 5

### Peptide Monomer Binding Affinity

The apparent dissociation constant (k_{d}) of the IRDye800-labeled WKG* peptide monomer was measured to provide an estimate of binding affinity. The IRDye800-labeled monomer was incubated with human HCC cells over a range of concentrations. Fluorescence intensities were measured with flow cytometry. Data for CD44 is shown in FIG. 4B. WKG*-IRDye800 was incubated with SK-Hep1 cells over concentrations ranging from 0-200 nM, and the fluorescence intensity was measured with flow cytometry. A k_{d}=43 nM was found.

### Example 6

### Peptide Monomer Stability in Serum

The optimized WKG* monomer labeled with IRDye800 was incubated in serum to determine half-life T₁/2. Stability in mouse serum over time was measured using analytical RP-HPLC. The relative concentration was calculated from the area-under-the-peak, and the intensities were fit to a first order kinetic model, and the serum half-life T_{1/2} was determined. Data for CD44 is shown in FIG. 4C. WKG*-IRDye800 was injected intravenously in live mice. Fluorescence intensity was measured time points ranging from 0-24 hours. A serum half-life of T_{1/2}=5.1 hours was found. This duration is sufficient to identify peak uptake in HCC tumor during in vivo imaging.

### Example 7

### Patient-Derived Xenograft (PDX) Tumors

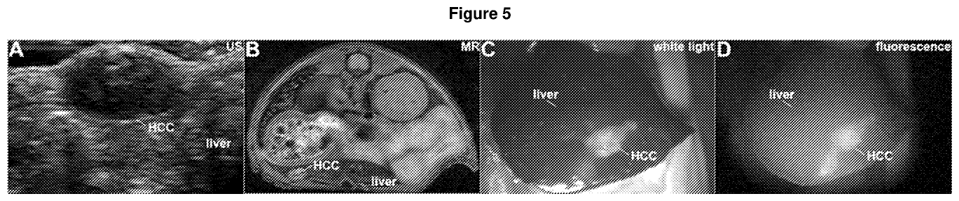

The Tissue Procurement Core (TPC) at the University of Michigan Rogel Cancer Center provided fresh de novo human HCC specimens. The specimens were implanted in the liver of NOD Cg-Prkdcll2rgSzJ (NSG) mice. In the mice, mutations in scid and a complete null allele of IL2rg^{null} result in extreme immunodeficiency. HCC tumor growth was e monitored weekly by ultrasound, FIG. 5A. MR and NIR fluorescence images were collected to confirm the orthotopic location of viable HCC tumors, FIG. 5B-D. After completion of imaging, the presence of human HCC tumor adjacent to mouse liver was confirmed by a human anti-cytokeratin stain using IHC. This pre-clinical model of HCC provides clinically relevant target expression levels and genetic heterogeneity seen in a broad patient population.

### Example 8

Validation of Specific Tumor Uptake of Multimer with In Vivo Imaging

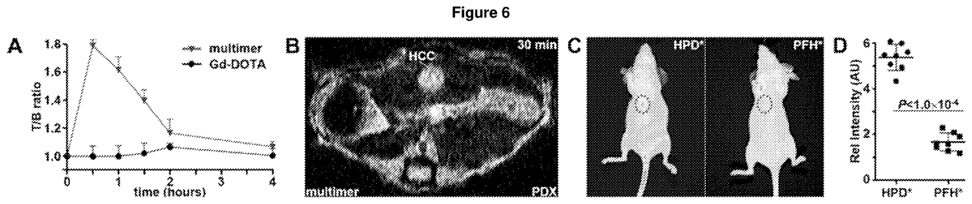

In vivo imaging was performed to assess HCC tumor uptake, biodistribution, and clearance of the optimized multimer. FIG. 6 shows data from MR images collected using the Gd-DOTA-labeled multimer and free Gd-DOTA (no peptides). Peak uptake was observed at 30 min post-injection, FIG. 6A. The target-to-background (T/B) ratio for the multimer was ~1.8-fold greater than that for monomer and free Gd-DOTA from the same tumor in n=3 animals. The target region was segmented using the Chan-Vese algorithm. The background region was defined by dilating the target region. Clearance of the MR signal occurred after ~4 hours post-injection. A T₁-weighted MR image of the HCC tumor collected at 30 min post-injection (peak uptake) is shown in FIG. 6B. Data from whole body NIR fluorescence images collected in vivo from the orthotopic HCC tumor following intravenous injection of the lead candidate EpCAM target and control peptides is shown in FIG. 6C. The mean value for the target peptide was significantly greater than that for control, FIG. 60. For the differences and variances shown in FIG. 60, the power is >95% to obtain P<0.01 for target versus control using n=8 mice per group.

### Example 9

### Peptide Multimer Biodistribution

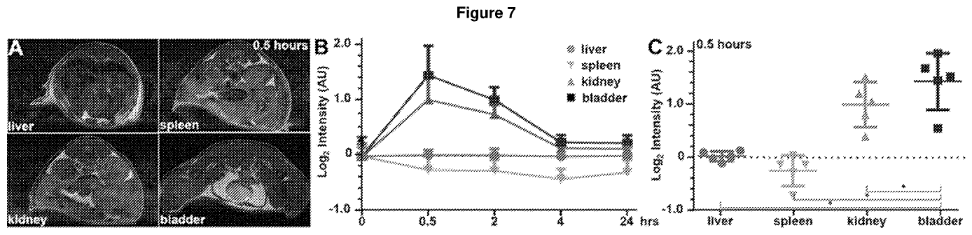

MR imaging was used to characterize the in vivo biodistribution of the Gd-DOT-labeled multimer configured with the optimized monomers over time in liver, spleen, kidney, and bladder. Segmentation was performed using the Chan-Vese algorithm. T₁-weighted MR images are shown in FIG. 7A. Peak signal was observed at 0.5 hours post-injection with return to baseline by ~4 hours, FIG. 7B. The increased signal in kidney and bladder was significantly higher than that for either liver or spleen, FIG. 7C. These findings support rapid peptide clearance, in particular, from the reticuloendothelial system (RES). For the differences and variances found in FIG. 7C, the power is >95% to obtain P<0.05 for target versus control using n=5 mice per group.

### Example 10

Validation of Target Expression Ex Vivo with Immunofluorescence

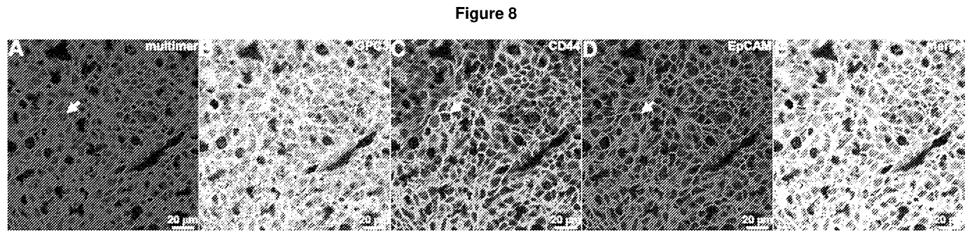

After completion of in vivo imaging, specific multimer binding to the PDX HCC tumor was validated ex vivo using confocal microscopy. These studies confirmed the in vivo imaging results and expression of the expected targets ex vivo. Data from a representative PDX specimen resected following completion of in vivo imaging is shown in FIG. 8. Increased fluorescence intensity is seen at the cell surface (arrow) for the IRDye800-labeled multimer binding to HCC tumor, FIG. 8A. Adjacent sections were stained with antibodies specific for GPC3, CD44, and EpCAM, FIG. 8B-D. Co-localization of multimer and antibody binding is shown on the merged image, FIG. 8E. A correlation of ρ=0.69, 0.65, and 0.70 was measured to characterize binding co-localization by the multimer with the GPC3, CD44, and EpCAM antibodies, respectively.

### Example 11

Validation of Target Expression Ex Vivo with Immunohistochemistry

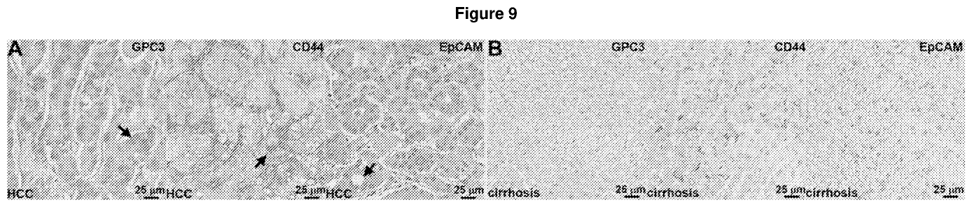

After completion of in vivo imaging, the PDX HCC tumor was resected and evaluated using immunohistochemistry (IHC). These studies confirmed expression of the expected targets ex vivo. Data from a representative PDX specimen resected following completion of in vivo imaging is shown in FIG. 9. Increased reactivity is seen at the cell surface (arrow) for GPC3, CD44, and EpCAM, FIG. 9A. By comparison, normal liver shows minimal reactivity for these targets, FIG. 9B. n=5 resected PDX HCC specimens were evaluated for each target.

### Example 12

### Binding of Cy5.5-Labeled Peptide Multimer to HCC Cells

HCC patient-derived cells (CCA-156) were generated from fresh, de novo tumor specimens obtained from patients undergoing liver transplant.

Fresh tissue was cut into ~1 mm² pieces in ice-cold DPBS. and 3-4 pieces were placed in one well of a 12-well plate. Minced tissue was cultured using Matrigel with growth media containing 10 μM ROCK inhibitor Y27632 (ATCC) and 5 μM TGF-β inhibitor (A83-01, Sigma-Aldrich) diluted to 8 mg/mL. An HCC cell line and a corresponding organoid were successfully generated. The cell line was denoted CCA-156 to reflect the specimen ID.

Approximately 10³ CCA-156 cells were grown on the cover glass in a 24-well plate to ~90% confluence. The cells were washed with PBS twice and incubated with 5 μM of either Cy5.5-labeled peptide multimer or peptide monomer for 5 min on ice. The cells were then washed with PBS for 3X, fixed with 4% paraformaldehyde (PFA) in PBS for 15 min, washed with PBS for twice, then mounted on glass slides with ProLong Gold reagent containing DAPI (Invitrogen).

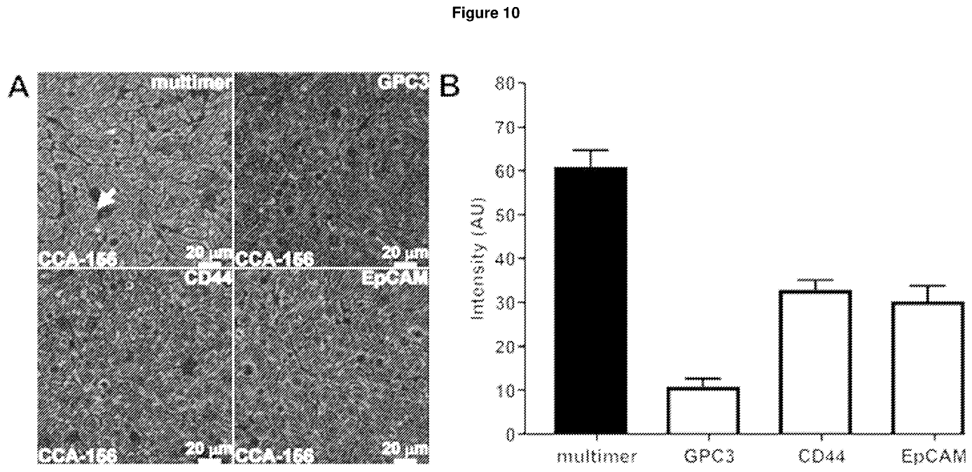

Strong fluorescence signal was observed for the peptide multimer versus the peptide monomers specific for GPC3, CD44, and EpCAM binding to the cell surface (arrow), FIG. 10A. The quantified intensities showed a 5.48, 1.84 and 2.00-fold increase for the multimer versus each monomer, respectively, FIG. 10B.

For a co-localization assay, patient-derived organoids were recovered from Matrigel and fixed by 4% PFA for 30 min. Permeabilization and blocking were performed using 5% serum (v/v) and 1% Triton X-100 (v/v) in PBS for 3 hours at room temperature. Organoids were then incubated with 1:200 dilution of primary recombinant rabbit anti-GPC3 antibody (#ab95363, Abcam), 1:500 dilution of primary recombinant mouse anti-CD44 antibody (#3570, Cell Signaling Technology), and AF594-labeled primary anti-EpCAM (#7319, Cell Signaling Technology) at 4° C. overnight. After washing with IF buffer (0.1% w/v BSA, 0.2% v/v Trito X-100 and 0.1% v/v TWEEN 20 in PBS), organoids was incubated with 1:500 dilution of AF488-labeled secondary goat anti-mouse IgG antibody (#ab150113, Abcam) and 1:500 dilution of AF532-labeled secondary goat anti-rabbit IgG antibody (Invitrogen) for 2 hours at room temperature. Organoids were then mounted on glass slides with ProLong Gold reagent containing DAPI (Invitrogen). Confocal fluorescence images were obtained on Leica Stellaris 5 (inverted) confocal microscope using a 63X oil-immersion objective. Fluorescence intensities were quantified using Leica LAS AF Lite software.

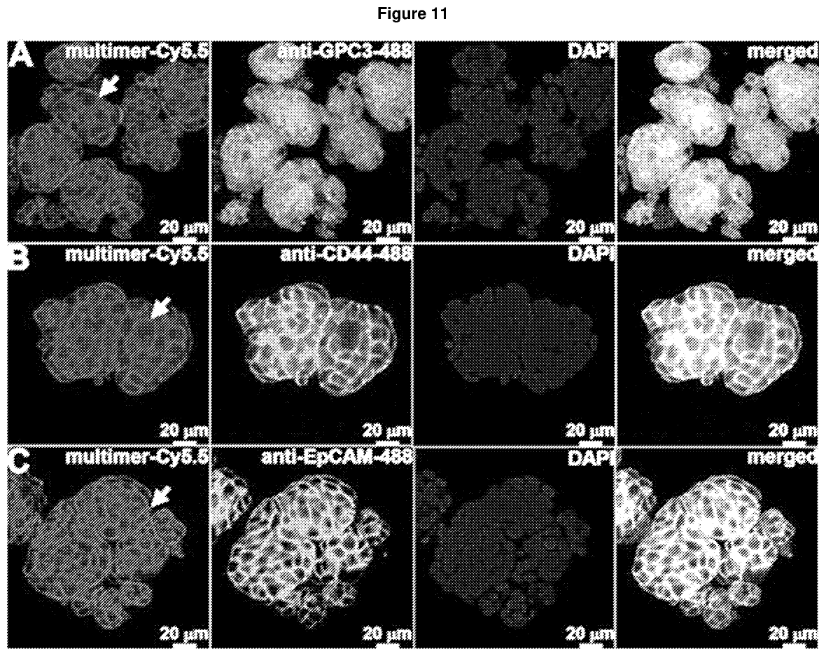

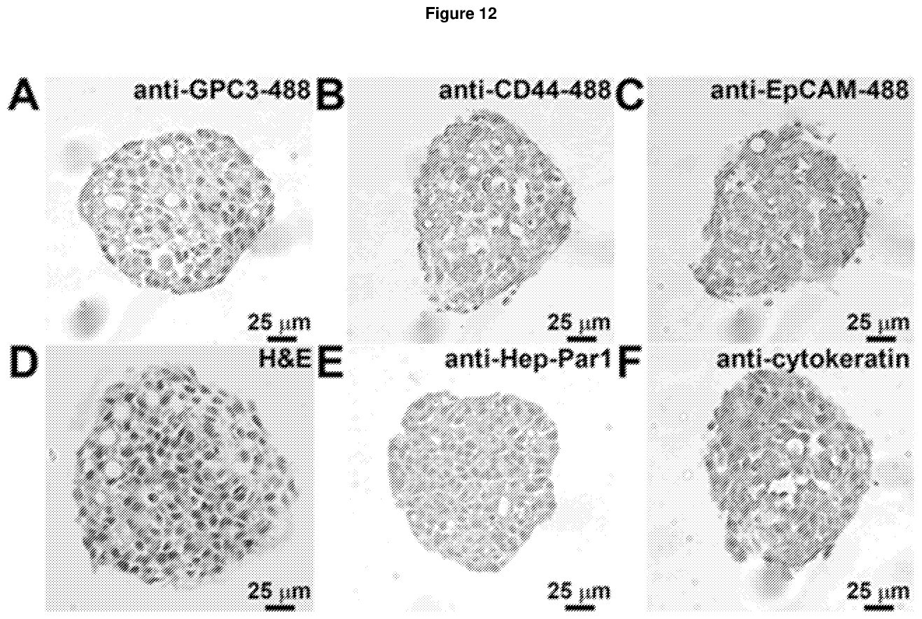

On immunofluorescence, co-localization was seen for binding by the Cy5.5-labeled peptide multimer and AF488-labeled antibodies specific for GPC3, CD44, and EpCAM, FIG. 11A-C. A correlation of r=0.74, 0.80, and 0.75, respectively, was measured on the merged images. Immunohistochemistry (IHC) was performed using anti-GPC3, anti-CD44, and anti-EpCAM, respectively, and all demonstrated strong reactivity to HCC, FIG. 12A-C. Features of tumor including the cytoplasmic appearance, steatosis, and pseudoglandular architecture were observed from the histology (H&E), FIG. 12D. Positive staining for Hep-Par-1 and anti-cytokeratin supports liver and human specific tissues, respectively, FIG. 12E,F.

### Example 13

### In Vivo Tumor Uptake of Gd-DOTA-Labeled Peptide Multimer

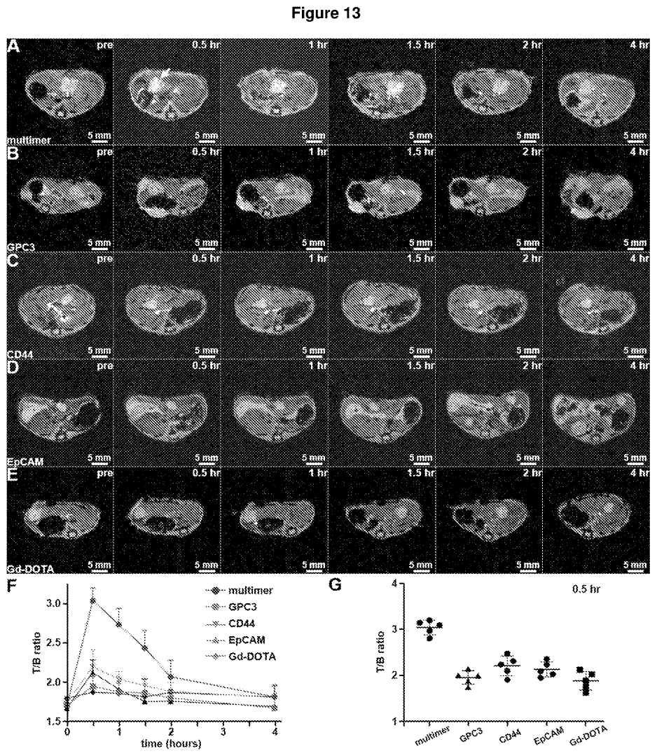

In vivo tumor uptake of the peptide multimer was evaluated in an orthotopic HCC PDX mouse model using T₁-weighted MR images. The Gd-DOTA labeled peptide multimer and peptide monomers specific for GPC3, CD44, and EpCAM were intravenously injected at a concentration of 300 μM in 200 μL of PBS via the tail vein of mice bearing orthotopic PDX tumors, FIG. 13A-E. Gd-DOTA (300 μM in 200 μL PBS) was also intravenously injected as a control. T1-weighted MR images were acquired using a 7T horizontal bore small animal magnet (SGRAD 205/120/HD/S, Agilent Technologies). MR image parameters include orientation=axial, echo time (TE): 10 ms, repetition time (TR): 717 ms, average=3, slices=35, thickness=0.5 mm, and display matrix (RO×PE)=256×128. MRI image construction was conducted by Matlab using scripts written and developed in-house. Images were collected prior to (pre) and at 0.5, 1, 1.5, 2, and 4 hours post-injection.

The tumor-to-background (T/B) ratio was measured and revealed peak tumor uptake of Gd-DOTA-labeled peptide multimer at 0.5 hours post-injection with clearance by ~4 hours, FIG. 13F. The mean value for the peptide multimer was greater than that for the GPC3, CD44, and EpCAM peptide monomers and for free Gd-DOTA, FIG. 13G.

### Example 14

### In Vivo Competition for Binding to GPC3, CD44, and EpCAM

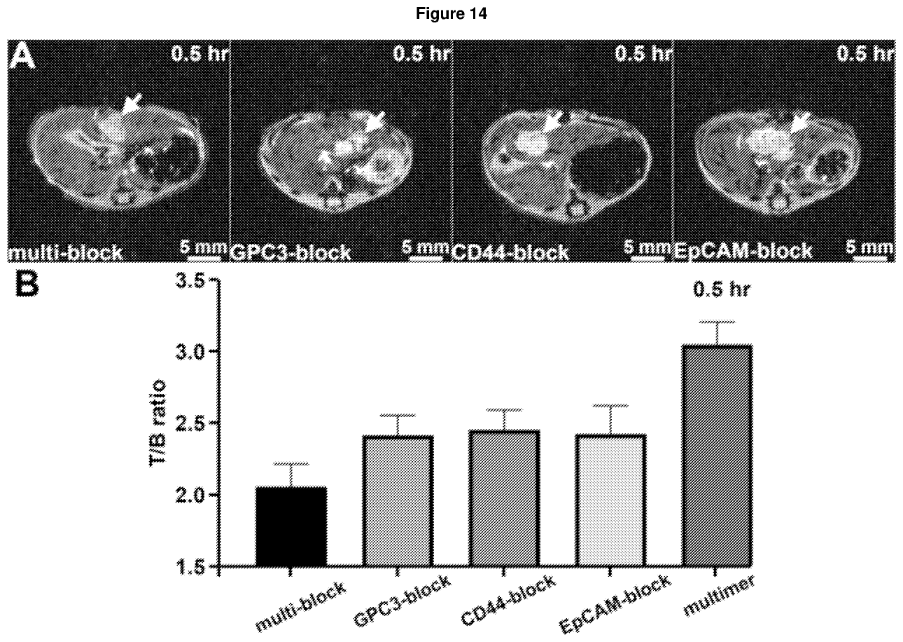

In vivo competition for binding to GPC3, CD44, and EpCAM was evaluated by administering unlabeled peptide monomers prior to Gd-DOTA labeled peptide multimer. Single unlabeled peptide monomers (ALL*, WKG*, and HPD*) at a concentration of 1.5 mM in 100 μL of PBS, or a mixture of all three peptide monomers (multi-block) at a concentration of 1 mM in 100 μL of PBS each were administered 30 minutes prior to the Gd-DOTA-labeled peptide multimer, FIG. 14A. T1-weighted MR images were collected at the peak uptake time of 0.5 hour after the peptide multimer injection. The T/B ratio of MR intensity was calculated using regions of interest, and the adjacent non-tumor region with equal area of the tumor region was defined as the background.

Quantified MRI intensities showed a significant reduction in the mean T/B ratio at 0.5 hour post-injection for all blocking groups versus that for the peptide multimer, FIG. 14B. Also, a decreased T/B ratio was observed with the multi-block group compared with single (GPC3, CD44, and EpCAM) block groups.

### Example 15

### Gd-DOTA-Labeled Peptide Multimer Biodistribution

Biodistribution of the Gd-DOTA-labeled peptide multimer in the orthotopic HCC PDX mouse model was evaluated using T₁-weighted MR images collected over time. Gd-DOTA-labeled peptide multimer was intravenously administrated and T1-weighted MR images were collected at different time intervals of pre, 0.5, 1, 1.5, 2, 4 hours. MR signal intensities were measured in abdominal major organs, including spleen, liver, and kidney.

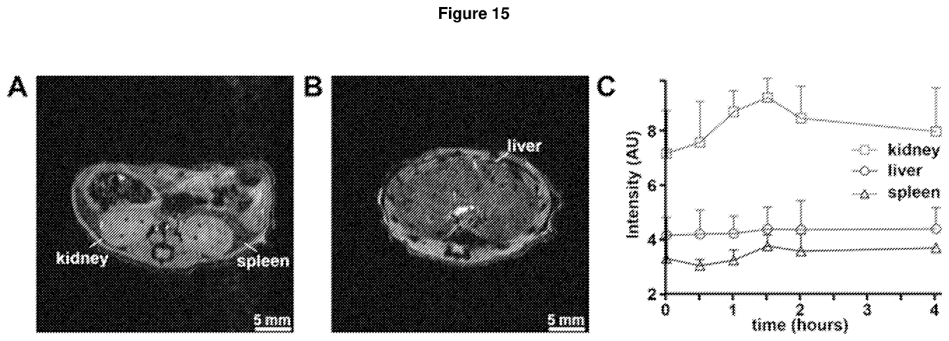

Greater MR signal was observed in the kidney than in either the liver or spleen from 0-4 hours after intravenous administration. Peak signal in kidney was visualized at 0.5 hours post-injection with return to baseline by ~4 hours to support rapid renal clearance, FIG. 15B.

### Example 16

### Serum Stability of the Gd-DOTA-Labeled Peptide Multimer

Serum stability was evaluated by diluting Gd-DOTA-labeled peptide multimer in fresh mouse serum 37° C. at a final concentration of 75 μM and incubating at 37° C. for 0, 0.5, 1, 1.5, 2, 3, 4, 6, 18 and 24 hours. Serum stability was measured by analytical HPLC (Waters 1525EF) using an analytical C18-column (XBridge™ C18, 5 μm, 4.6×150 mm2) with a water (0.1% TFA)-acetonitrile (0.1% TFA) gradient. The flow rate was 1.0 mL/min, and the retention time was ~50 min. The relative peptide concentrations were calculated by the area under the peak.

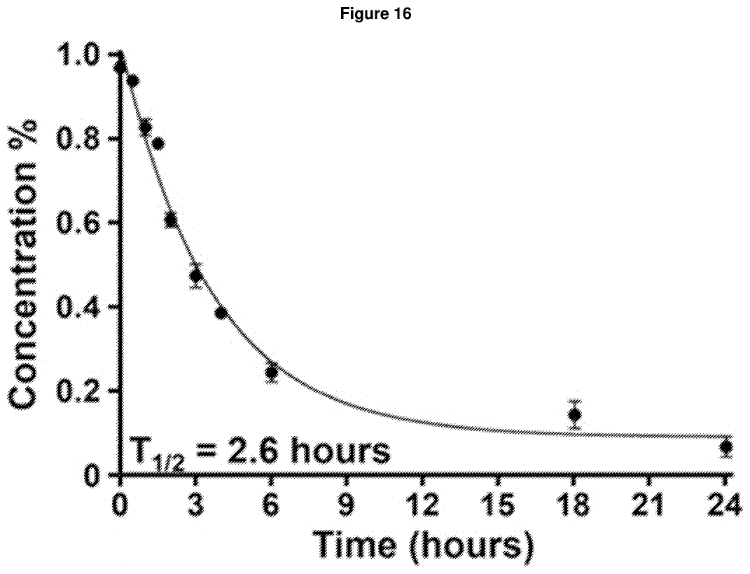

A half-life of T_{1/2}=2.6 hours was measured for the Gd-DOTA-labeled peptide multimer, FIG. 16.

### Example 17

### Tumor Binding by Cy5.5-Labeled Peptide Multimer

Orthotopic HCC PDX tumors were resected and sectioned for immunofluorescence.

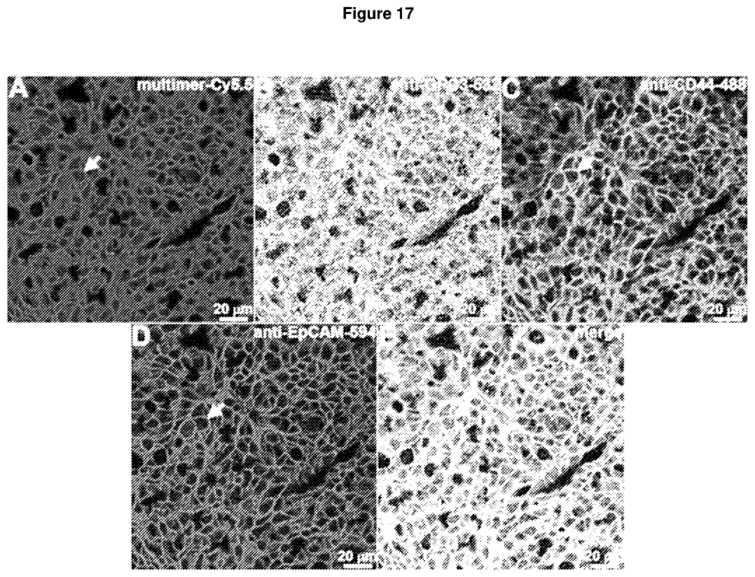

Binding by the Cy5.5-labeled peptide multimer co-localized (arrow) with that for fluorescently-labeled antibodies specific for GPC3, CD44, and EpCAM, FIG. 17A-D. A correlation of r=0.69, 0.65, and 0.70, respectively, was measured on the merged images, FIG. 17E.

### Example 18

### Animal Necropsy

Healthy mice were euthanized at 48 hours post-injection of Gd-DOTA-labeled peptide multimer. Vital organs were collected, and histology was evaluated by an expert pathologist.

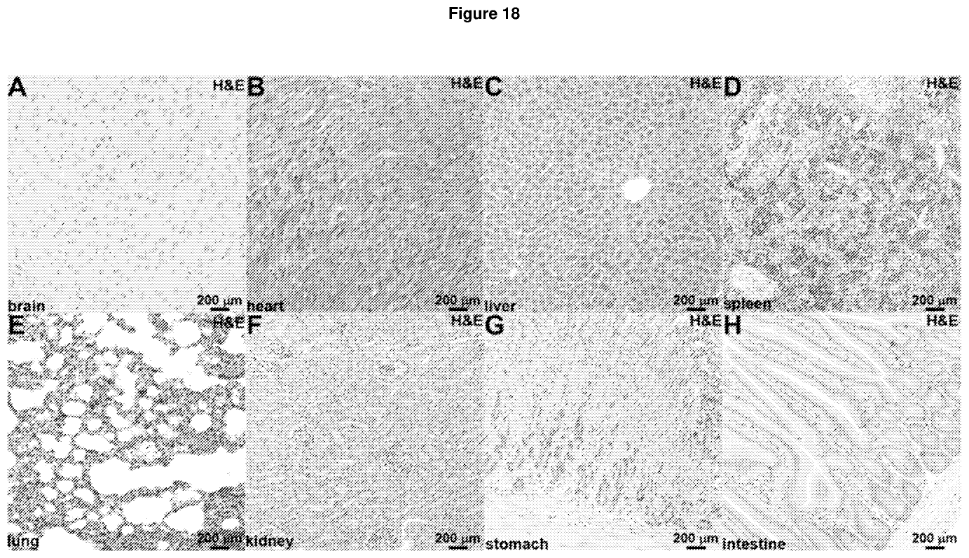

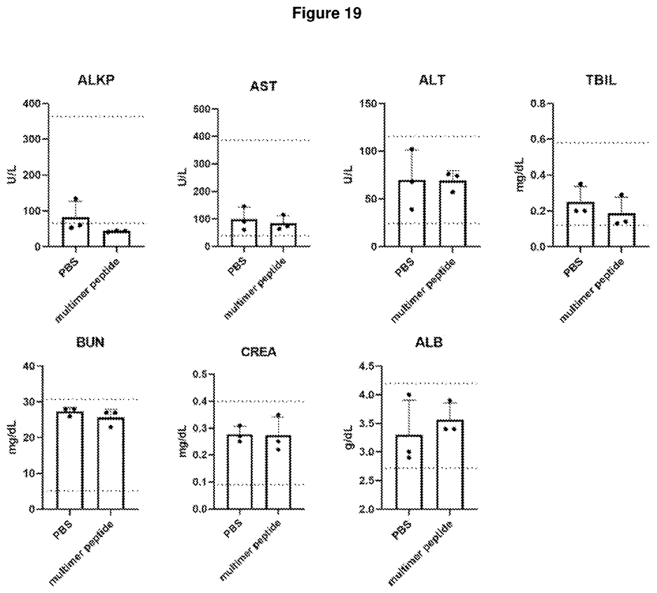

No signs of acute toxicity were found in the brain, heart, liver, spleen, lung, kidney, stomach and intestine, FIG. 18. Serum was also collected for blood chemistry test, and no acute toxicity was observed, FIG. 19.

### Example 19

### Binding of Cy5.5-Labeled Peptide Multimer to Human HCC Specimens Ex Vivo

Binding of Cy5.5-labeled peptide multimer was evaluated using human HCC specimens ex vivo. All experiments using human tissues were approved by the Michigan Medicine IRB (HUM00122873). A tissue microarray (TMA) was generated from n=120 liver specimens provided by the archived tissue bank in the Department of Pathology. Paraffin-embedded (FFPE) section slide was deparaffinized and conducted through antigen retrieval. Blocking procedure was performed with 5% goat serum for 1 hour at room temperature. The slide was first stained with the Cy5.5 labeled peptide multimer at 5 μM for 5 min at room temperature. The section was then incubated with primary antibodies using 1:200 dilution of primary recombinant rabbit anti-GPC3 antibody (#ab95363, Abcam), 1:500 dilution of primary recombinant mouse anti-CD44 antibody (#3570, Cell Signaling Technology), and AF594-labeled primary anti-EpCAM (#7319, Cell Signaling Technology) overnight at 4° C. After washing with 2% BSA in PBST, section was incubated with 1:500 dilution of AF488-labeled secondary goat anti-mouse IgG antibody (#ab150113, Abcam) and 1:500 dilution of AF532-labeled secondary goat anti-rabbit IgG antibody (#A-11029, Life Technologies) for 1 hour at room temperature, and mounted with Prolong Gold reagent containing DAPI at last (Invitrogen). The fluorescence images were collected using Leica Stellaris 5 (inverted), excitation at Aex=670 nm for the peptide multimer, and λex=488, 532, 594 nm for CD44, GPC3 and EpCAM antibody, respectively. Fluorescence intensities were quantified using Leica LAS AF Lite software. All slides were evaluated by an expert liver pathologist.

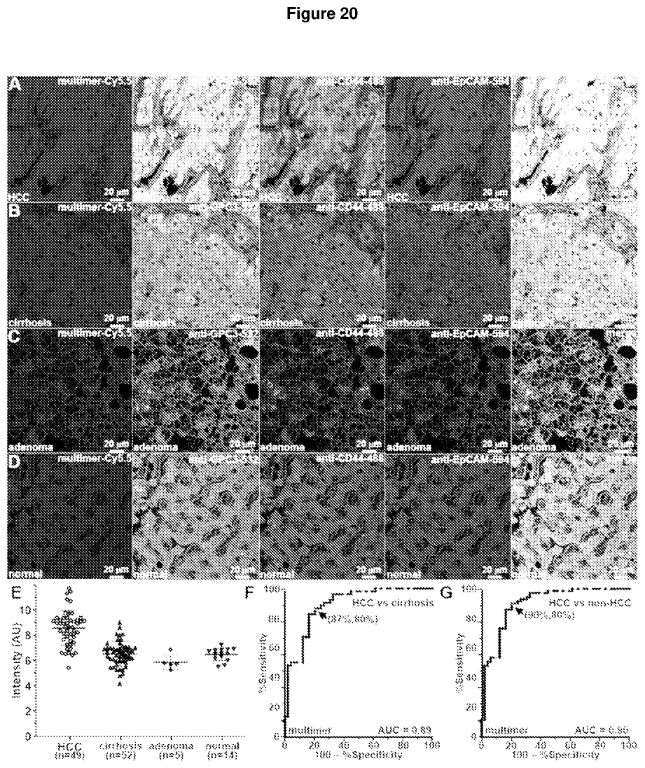

Quantified fluorescence intensities showed the mean value for HCC was significantly greater than that for cirrhosis, adenoma, and normal, FIG. 20E. The ROC curves generated showed 87% sensitivity and 80% specificity for distinguishing HCC from cirrhosis with AUC=0.89, and 90% sensitivity and 80% specificity for distinguishing HCC from non-HCC with AUC=0.90, FIG. 20F,G.

The Cy5.5-labeled peptide multimer provided improved performance for HCC detection with higher sensitivity and specificity compared with the peptide monomers.

### Example Summary

The peptide multimers and methods provided herein address the inability of conventional imaging to distinguish the pathology of indeterminant liver nodules <2 cm in size. The peptide multimers provided herein detect three tissue targets concurrently to identify early stage HCC using in vivo imaging. This multiplexed approach addresses genetic heterogeneity and molecular variability of individual tumor cells, and accounts for the influence of the tumor microenvironment (TME).

We claim:

1. A peptide multimer comprising a GPC3-specific peptide, a CD44-specific peptide and an EpCAM-specific peptide,
wherein the GPC3-specific peptide comprises the amino acids ALLANHEELFQT (SEQ ID NO: 1), ALLANHEELF (SEQ ID NO: 2), GLHTSATNLYLH (SEQ ID NO: 3), SGVYKVAYDWQH (SEQ ID NO: 4), or VGVESCASRCNN (SEQ ID NO: 5),
wherein the CD44-specific peptide comprises the amino acids WKGWSYLWTQQA (SEQ ID NO: 6), and
wherein the EpCAM-specific peptide comprises the amino acids HPDMFTRTHSHN (SEQ ID NO: 7), HGLHSMHNKLQD (SEQ ID NO: 8), GKPAVHYIHLRH (SEQ ID NO: 9), or HPFLHWNYGQRT (SEQ ID NO: 10).

2. The peptide multimer of claim 1 comprising:
at least one detectable label attached to a peptide of the multimer,
at least one therapeutic moiety attached to a peptide of the multimer, or
at least one detectable label attached to a peptide of the multimer and at least one therapeutic moiety attached to a peptide of the multimer.

3. The peptide multimer of claim 2 wherein the detectable label is detectable by optical, photoacoustic, ultrasound, positron emission tomography or magnetic resonance imaging.

4. The peptide multimer of claim 3 wherein the label detectable by optical imaging is fluorescein isothiocyanate (FITC), Cy5, Cy5.5 or IRdye800.

5. The peptide multimer of claim 4 wherein the label is IRdye 800.

6. The peptide multimer of claim 3 wherein the label detectable by magnetic resonance imaging is gadolinium, Gd-DOTA or an iron oxide nanoparticle.

7. The peptide multimer of claim 6 wherein the label is Gd-DOTA.

8. The peptide multimer of claim 1 wherein the multimer form of the peptide is assembled with a tri-lysine linker.

9. The peptide multimer of claim 2 wherein the detectable label is attached to the peptide by a peptide linker.

10. The peptide multimer of claim 9 wherein a terminal amino acid of the linker is lysine.

11. The peptide multimer of claim 10 wherein the linker comprises the sequence GGGSK set out in SEQ ID NO: 11.

12. The peptide multimer of 1, 2, 3, 4, 5, 6, 7, 8, 9, 10 or 11 comprising at least one therapeutic moiety attached to the peptide.

13. The peptide multimer of claim 12 wherein the therapeutic moiety is chemotherapeutic agent.

14. The peptide multimer of claim 12 wherein the therapeutic moiety is a polymeric nanoparticle or micelle.

15. The peptide multimer of claim 13 wherein the micelle is an octadecyl lithocholate micelle.

16. The peptide multimer of claim 15 wherein the nanoparticle or micelle is pegylated.

17. The peptide multimer of claim 13 wherein the nanoparticle or micelle encapsulates sorafenib, carboplatin, paclitaxel, cisplatin, 5-fluorouracil (5-FU), oxaliplatin, capecitabine, irinotecan or chlorambucil.

18. A composition comprising the peptide multimer of claim 1, 2, 3, 4, 5, 6, 7, 8, 9, 10, 11, 12, 13, 14, 15, 16 or 17 and a pharmaceutically acceptable excipient.

19. A method for detecting epithelial cell-derived cancer cells in a patient comprising the steps of administering the peptide multimer of claim 1, 2, 3, 4, 5, 6, 7, 8, 9, 10 or 11 to the patient and detecting binding of the peptide multimer to cells.

20. A method for treating an epithelial cell-derived cancer comprising administering to a patient a peptide multimer of claim 12, 13, 14, 15, 16 or 17.

21. A method of determining the effectiveness of a treatment for an epithelial cell-derived cancer in a patient comprising the step of administering the peptide multimer of claim 1, 2, 3, 4, 5, 6, 7, 8, 9, 10 or 11 to the patient, visualizing a first amount of cells labeled with the peptide multimer, and comparing the first amount to a previously-visualized second amount of cells labeled with the peptide multimer,
wherein a decrease in the first amount cells labeled relative to the previously-visualized second amount of cells labeled is indicative of effective treatment.

22. The method of claim 21 further comprising obtaining a biopsy of the cells labeled by the peptide multimer.

23. A method for delivering a therapeutic moiety to epithelial cell-derived cancer cells of a patient comprising the step of administering the peptide multimer of claim 12 to the patient.

24. The method of claim 19 wherein the cancer cells are hepatocellular carcinoma (HCC) cells, intrahepatic cholangiocarcinoma (ICC) cells, breast cancer cells, colon cancer cells, gastric cancer cells, ovarian cancer cells, cervical cancer cells, and basal cell carcinoma of the skin cells.

25. The method of claim 20 wherein the epithelial cell-derived cancer is hepatocellular carcinoma (HCC), intrahepatic cholangiocarcinoma (ICC), breast cancer, colon cancer, gastric cancer, ovarian cancer, cervical cancer, and basal cell carcinoma of the skin.

26. The method of claim 21 wherein the epithelial cell-derived cancer is hepatocellular carcinoma (HCC), intrahepatic cholangiocarcinoma (ICC), breast cancer, colon cancer, gastric cancer, ovarian cancer, cervical cancer, and basal cell carcinoma of the skin.

27. The method of claim 22 wherein the epithelial cell-derived cancer is hepatocellular carcinoma (HCC), intrahepatic cholangiocarcinoma (ICC), breast cancer, colon cancer, gastric cancer, ovarian cancer, cervical cancer, and basal cell carcinoma of the skin.

28. The method of claim 23 wherein the cancer cells are hepatocellular carcinoma (HCC) cells, intrahepatic cholangiocarcinoma (ICC) cells, breast cancer cells, colon cancer cells, gastric cancer cells, ovarian cancer cells, cervical cancer cells, and basal cell carcinoma of the skin cells.

29. A kit for administering the composition of claim 18 to a patient in need thereof, said kit comprising the composition of claim 18, instructions for use of the composition and a device for administering the composition to the patient.


SEQUENCE LISTING (11 sequences)

SEQ ID NO: 1 - protein, 12 aa, synthetic construct
  source 1..12: note = GPC3-specific peptide
ALLANHEELFQT

SEQ ID NO: 2 - protein, 10 aa, synthetic construct
  source 1..10: note = GPC3-specific peptide
ALLANHEELF

SEQ ID NO: 3 - protein, 12 aa, synthetic construct
  source 1..12: note = GPC3-specific peptide
GLHTSATNLYLH

SEQ ID NO: 4 - protein, 12 aa, synthetic construct
  source 1..12: note = GPC3-specific peptide
SGVYKVAYDWQH

SEQ ID NO: 5 - protein, 12 aa, synthetic construct
  source 1..12: note = GPC3-specific peptide
VGVESCASRCNN

SEQ ID NO: 6 - protein, 12 aa, synthetic construct
  source 1..12: note = CD44-specific peptide
WKGWSYLWTQQA

SEQ ID NO: 7 - protein, 12 aa, synthetic construct
  source 1..12: note = EpCAM-specific peptide
HPDMFTRTHSHN

SEQ ID NO: 8 - protein, 12 aa, synthetic construct
  source 1..12: note = EpCAM-specific peptide
HGLHSMHNKLQD

SEQ ID NO: 9 - protein, 12 aa, synthetic construct
  source 1..12: note = EpCAM-specific peptide
GKPAVHYIHLRH

SEQ ID NO: 10 - protein, 12 aa, synthetic construct
  source 1..12: note = EpCAM-specific peptide
HPFLHWNYGQRT

SEQ ID NO: 11 - protein, 5 aa, synthetic construct
  source 1..5: note = Linker
GGGSK